(19) United States
(12) Patent Application Publication
(10) Pub. No.: US 2009/0270884 A1
(43) Pub. Date: Oct. 29, 2009

(54) Mesh Dispensing Device Having a Closed Loop of Backing Film

(75) Inventor: Gregory HAKE, Otsego, MN (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: CLOSURE MEDICAL CORPORATION, Raleigh, NC (US)

(21) Appl. No.: 12/109,402

(22) Filed: Apr. 25, 2008

(51) Int. Cl. A61B 17/03; A61B 19/00

(52) U.S. Cl. 606/139; 606/215; 156/429

(57) ABSTRACT

A mesh dispensing device includes a housing having a substantially enclosed interior and an opening; a plurality of routing guide elements mounted in the interior of the housing; and a closed loop of backing film having a defined circumference. The closed loop of backing film is routed around at least a portion of the plurality of routing guide elements to define a path along which the closed loop of backing film moves. The device also includes a defined length of mesh that is detachably adhered to the closed loop of backing film and an opening guide element mounted near the opening of the housing. The opening guide element receives mesh that has been detached from the closed loop of backing film for dispensing.

### FIELD

The present invention relates to a dispensing device for applying/dispensing a mesh.

### BACKGROUND

Presently, patients undergoing surgical procedures requiring long incisions may require their incisions to be closed using sutures, staples, and/or adhesive strips. All of these closing methodologies may have difficulties, to varying degrees, with patient comfort, speed (for the clinician), clinical outcome (suture related infection/irritation), recovery time and cosmesis.

Skin closure strips, such as conventional adhesive bandages, are utilized for closure of relatively superficial skin wounds. However, the contact adhesives that are used with such strips typically retain adhesion for no more than a day or two and can lose adhesion quickly in the presence of moisture, for example, perspiration.

While improved materials and methods for wound approximation are generally known, for example, co-assigned U.S. Patent Application Publication No. 2006/0009099, these improved materials may be more generally accepted and more widely used if dispensing devices adapted to the unique characteristics of these materials were available. Therefore, a need continues to exist for devices and methods for dispensing materials useful in wound approximation and tissue bonding to provide a wider range of applications for these materials, from external to internal use, and from essentially non-biodegradable (where the materials are removed from the application site) to biodegradable (where the materials are not directly removed from the application site and degrade over time).

### SUMMARY

Devices and methods for dispensing materials useful in wound approximation and tissue bonding are provided. The device is adapted to characteristics of the materials used in wound approximation and tissue bonding. Methods of tissue bonding using the device are also provided.

The dispensing device herein disclosed may be used to dispense a material suitable for bonding a variety of tissue ranging from hard tissue (such as bone) to soft tissue (such as skin, organs, mucous membranes, and the like). The tissue can be either internal or external.

The present invention includes many aspects and features.

In a first aspect of the invention, a mesh dispensing device includes a housing having a substantially enclosed interior and an opening, a plurality of routing guide elements mounted transversely in the interior of the housing, and a closed loop of backing film having a defined circumference. The closed loop of backing film is routed around at least a portion of the plurality of routing guide elements to define a path along which the closed loop of backing film moves. The device further includes a defined length of mesh that has an adhesive substance applied over at least a portion of a side of the mesh. In addition, the mesh is detachably adhered to the closed loop of backing film via the adhesive side of the mesh. The device also includes an opening guide element mounted transversely near the opening of the housing. The opening guide element receives mesh that has been detached from the closed loop of backing film, such that the adhesive side of the mesh is presented at the opening of the housing for dispensing.

In a feature of this aspect, the opening guide element consists of a roller rotatably mounted at the opening. The rotatably mounted roller of the guide element cooperates with a guide element of the plurality of routing guide elements to separate the mesh from the closed loop of backing film and present the adhesive side of the mesh at the opening of the housing. In another feature, the plurality of routing guide elements are rotatably mounted rollers.

In an additional feature, spacing between the opening guide element and the plurality of routing guide elements is sufficient to enable a viewing window to be formed in the housing between the opening guide element and the plurality of routing guide elements. In a further feature, the plurality of routing guide elements and the opening guide element may consist of rollers that are the same size such that they are interchangeable. In yet another feature, the defined circumference of the closed loop of backing film is approximately about the same as the defined length of mesh.

In a still further feature, the plurality of routing guide elements consists of four routing guide elements. The plurality of routing guide elements may also consist of more or less than four routing guide elements. In addition, the housing includes two interchangeable side members that are joined to form the housing.

In a second aspect of the invention, a mesh dispensing device includes a housing having an opening, a plurality of routing guide elements mounted transversely in the housing, and a closed loop of backing film having a defined circumference. The closed loop of backing film is routed around at least a portion of the plurality of routing guide elements to define a convoluted path along which the closed loop of backing film moves. The device also includes a defined length of mesh that has an adhesive substance applied over at least a portion of a side of the mesh. The mesh is detachably adhered to the closed loop of backing film via the adhesive side of the mesh. Further, the mesh is separated from the closed loop of mesh and backing film such that the adhesive side of the separated mesh is presented to the opening of the housing for dispensing.

In features of this aspect, the convoluted path consists of a V-shaped path and the plurality of routing guide elements consist of rotatably mounted rollers. With regard to this feature, each roller of the plurality of rollers is the same size such that the rollers are interchangeable. In another feature, the defined circumference of the closed loop of backing film is approximately about the same as the defined length of mesh.

In an additional feature, the closed loop of backing film is routed around all of the plurality of routing guide elements. In yet another feature, the plurality of routing guide elements consists of four routing guide elements. In further features, the housing includes two interchangeable side members that are assembled to form the housing and the mesh further includes a polymerization initiator.

In a third aspect of the invention, a method for tissue bonding includes providing a dispensing device wherein the dispensing device includes a housing having an opening, a plurality of routing guide elements mounted transversely in the housing, and a closed loop of backing film having a defined circumference. The closed loop of backing film is routed around at least a portion of the plurality of routing guide elements to define a path along which the closed loop of backing film moves. The device also includes a defined length of mesh having a pressure sensitive adhesive substance applied over at least a portion of a side of the mesh. In addition, the mesh is detachably adhered to the closed loop of backing film via the adhesive side of the mesh. The device further includes an opening guide element mounted transversely in the housing. The opening guide element receives mesh that has been separated from the closed loop of backing film, such that the adhesive side of the mesh is presented at the opening of the housing for dispensing. The method also includes contacting a tissue surface in need of bonding, wherein at least a portion of the pressure sensitive adhesive side of the mesh is positioned between the tissue surface and the opening guide element; adhering the at least a portion of the pressure sensitive adhesive side of the mesh to the tissue surface; operating the dispensing device along the tissue surface; and dispensing the mesh from the opening of the device; and bonding the tissue surface.

In a feature of this aspect, the mesh is impregnated with a polymerization initiator. In another feature, the step of bonding the tissue surface by dispensing the mesh from the opening of the dispensing device along the tissue surface also includes approximating the tissue surface in need of bonding. In an additional feature, the method further includes the steps of applying a polymerizable adhesive composition over and substantially covering at least a portion of the mesh and allowing the polymerizable adhesive composition to permeate into and under the mesh and polymerize to form a composite structure bonded to the tissue surface.

### BRIEF DESCRIPTION OF THE DRAWINGS

FIG. 1 is a perspective view of a mesh dispensing device in accordance with a preferred embodiment of the present invention;

FIG. 2 is a perspective view of the mesh dispensing device of FIG. 1 with a side member thereof exploded to better show an inside of the dispensing device;

FIG. 3 is a top plan view of the mesh dispensing device of FIG. 1 with a side member thereof removed;

FIG. 4 is a perspective view of internal components of the dispending device of FIG. 1;

FIG. 5 is a perspective view of a mesh dispensing device in accordance with an alternative embodiment of the present invention;

FIG. 6 is a perspective view of the mesh dispensing device of FIG. 5 with a side member thereof removed to better show an inside of the dispensing device;

FIG. 7 is a top plan view of the mesh dispensing device of FIG. 5 with the side member thereof removed; and

FIG. 8 is a perspective view of a mesh dispensing device in accordance with a second alternative embodiment of the present invention.

### DETAILED DESCRIPTION

Devices and methods for dispensing materials useful in wound approximation and tissue bonding are provided. The device is adapted to characteristics of the materials used in wound approximation and tissue bonding, in particular, components of composite products used in tissue bonding.

A dispensing device configured to operate with an adhesive backed mesh and backing film for tissue bonding is provided. The dispensing device provides for pay-out of a desired length of the adhesive backed mesh with little to no friction. The dispensing device is able to accommodate varying lengths of mesh, including long strips of mesh, which is particularly advantageous for deployment of mesh when a wound is long. In addition, the device is adapted to be able to deploy mesh around curved surfaces, which are often present in wound approximation. The device minimizes, prevents, or eliminates distortion of the mesh prior to application to the wound site, which distortion can decrease the width of the mesh thereby preventing it from covering the full width of the wound and can prevent the adhesive backing from holding the mesh securely to a patient. Moreover, the dispensing device herein disclosed is configured to operate with one hand in a “pushing” mode or direction to provide essentially an unobstructed view of the wound site during use. By means of the “forward” mode or direction, the adhesive side of the mesh is dispensed and presented via passing beneath the device. For example, the mesh may be applied to a longitudinal wound starting at a region proximal to the dispensing device and moving the dispensing device along the longitudinal dimension of the wound towards a distal region. The mesh is dispensed by passing under the dispensing device leaving an applied mesh on the wound site starting at the proximal region and extending towards the distal region such that the view of the distal portion of the wound is substantially unobstructed by the dispensing device as it is moved along the longitudinal dimension of the wound. Such mode of operation may provide for improved surgical and clinical application of the aforementioned composite tissue bonding products by enabling an operator to clearly see the path of the wound line thus ensuring proper alignment of the mesh with the wound line and by enabling the operator to have a free hand for wound approximation. While it is preferred to operate the device in a pushing mode, it will be apparent that the device also may be used in a more conventional pulling mode. In addition, after mesh is applied to a wound, a polymerizable adhesive composition may be applied over the mesh to form a composite structure bound to the tissue surface of the wound.

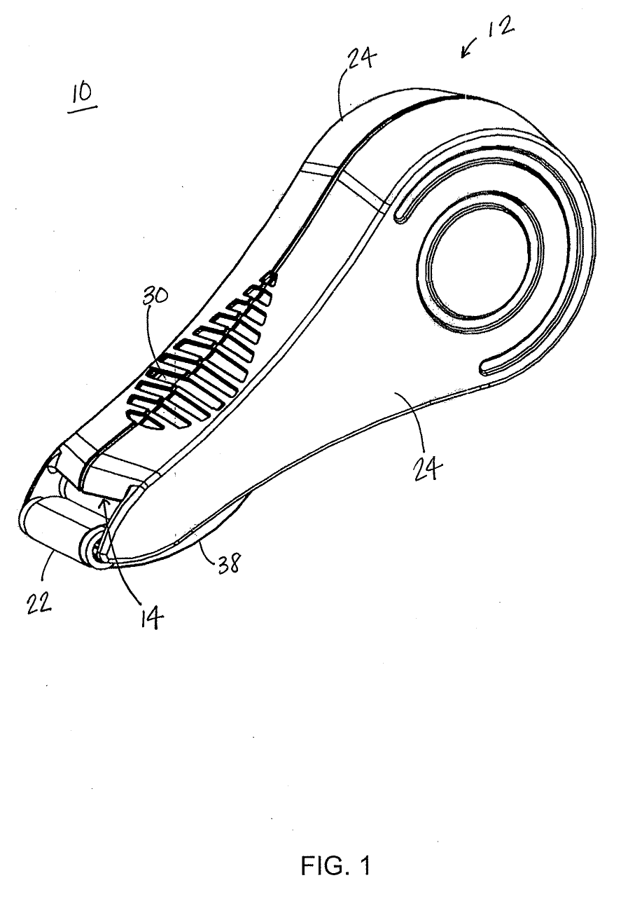

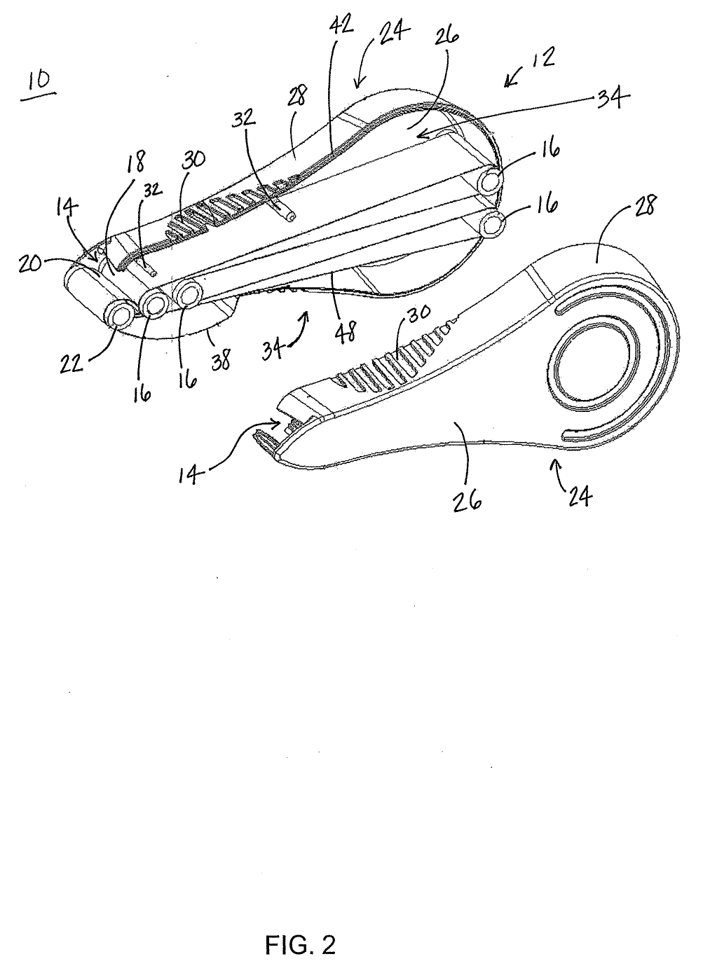

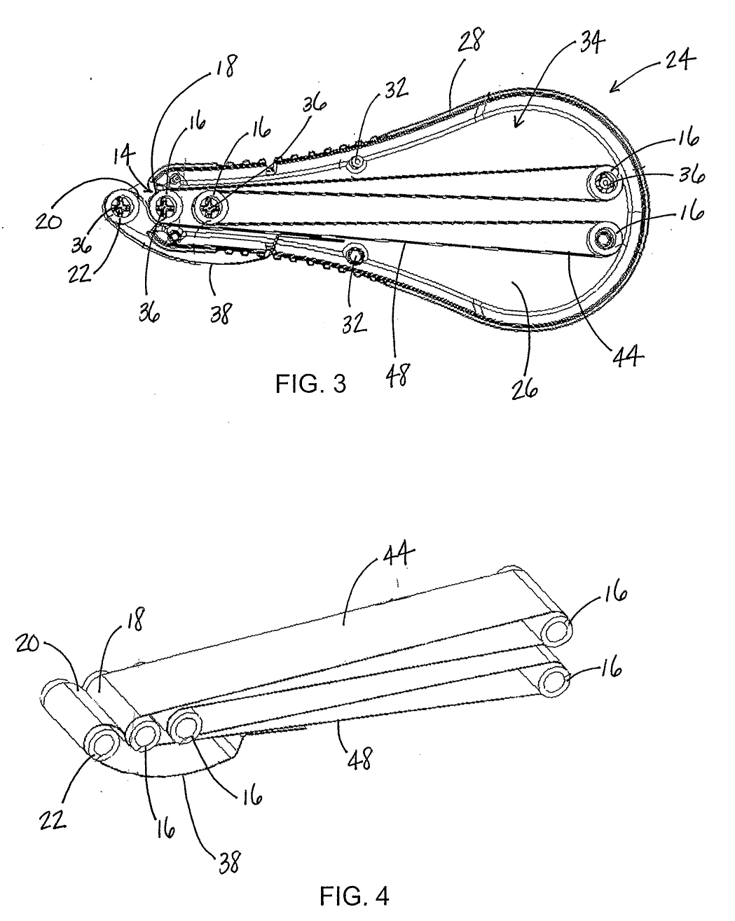

Referring to the figures, FIGS. 1-3 show a mesh dispensing device in accordance with a preferred embodiment of the present invention. More particularly, FIG. 1 is a perspective view of the mesh dispensing device, FIG. 2 is a partially exploded perspective view of the mesh dispensing device, and FIG. 3 is a top plan view of the mesh dispensing device with a side member of the device removed to better show an inside of the device.

With reference to paragraphs FIGS. 1 and 2, the mesh dispensing device 10 comprises an ergonomically shaped housing 12 having an opening 14 formed in a proximal end thereof, a plurality of routing guide elements 16 mounted in the housing 12, a closed loop of backing film 18 routed around at least a portion of the plurality of routing guide elements 16, mesh 20 detachably adhered to the closed loop of backing film 18; and optionally, an opening guide element 22 mounted at the opening 14 of the housing.

With reference to FIG. 2, the device housing 12 comprises two side members 24 that are joined to form the housing 12. Each side member 24 includes a side wall 28 and a perimeter wall extending around and away from a periphery of the side wall 26. The perimeter wall 28 extends substantially completely around the periphery of the side wall 26 except for a spaced area in the perimeter wall 28 that forms the opening 14 of the housing 12 with the spaced area of the other side member 24 when the side members 24 are joined. Ridges 30 may be formed in portions of the perimeter wall 28 and/or portions of the side wall 26 for improving a clinician's ability to hold the device 10 during use. In addition, other contoured indentations (not shown) may be included in the side walls 26 or perimeter walls 28 of the housing 12.

Mating connection members 32 project orthogonally from the side walls 26 of the housing 12 into an interior 24 of the housing 12. In the present embodiment, the mating connection members 32 are projections that fit together in a friction fit to join the side members 24 thereby forming the housing 12. As such, the projection 32 of one side member 24 consists of a solid projection member and the mating projection 32 of the other side member 24 consists of a sleeve into which the solid projection is inserted in a friction fit. Alternative conventional connection members may be used to join the side members 24 of the housing 12 together, including, but not limited to, screws, adhesives, and snap fit connectors. In addition, a snap fit closure mechanism (not shown) may be included around the edges of the perimeter walls 28, i.e., one side member 24 may have a groove formed around the edge of its perimeter wall 28 configured to accept a mating ledge formed around the edge of the perimeter wall 28 of the other side member 24. Alternatively, it is also contemplated that a recessed ledge 42 may be formed around the edge of the perimeter wall 28 of both side members 24. Although the recessed ledges 42 may not perform a connecting function, they would abut one another and provide an aesthetically pleasing appearance to the housing 12 when it is assembled. It is further contemplated that the two side members 24 of the housing 12 may be identical to one another, and therefore be interchangeable, to promote ease and convenience of manufacture and provide cost savings. The housing 12 may be constructed of plastic, metal, or combinations thereof.

It is preferred that the mesh dispensing device 10 is a disposable device, and therefore once the housing 12 is assembled, it will not be disassembled. However, the device 10 may be disassembled by disconnecting the side members 24 from one another for repair or replacement of various internal components.

With reference to FIG. 3, the plurality of routing guide elements 16 are mounted to the side members 24 of the housing 12 (shown in FIG. 2) in orthogonal relation thereto such that the routing guide elements 16 transverse the interior 34 of the housing 12. In the present embodiment, the routing guide elements 16 are rotatably mounted rollers. Corresponding bosses 36 extending from the side walls 26 of the housing 12 serve as axles about which the rollers 16 rotate. Such arrangement ensures that each of the rollers 16 is operatively connected to each of the side members 24. While bosses 36 serve as roller axles in the present embodiment, it is contemplated that conventional axle structures including pins, bars, shafts, and the like may also serve as axles for the rollers 16. Further, it is contemplated that mating projections similar to the connection members 32 may be utilized as the roller axles to provide additional points of connection between the side members 24 of the housing 12. The present embodiment includes four rollers 16; however, it is contemplated that the device 10 may include more or less rollers 16 and still be within the scope of the present invention. The rollers 16 may be constructed of plastic, metal, or combinations thereof. For example, the opening guide element may be Teflon®, polyethylene, ultra-high molecular weight polyethylene or stainless steel.

Alternatively, the routing guide elements 16 may be fixedly, rather than rotatably, mounted to the housing 12 provided that the surfaces of the routing guide elements 16 are low friction. Low surface friction may include the application of lubricants, surface finishes, coatings, and/or other treatments suitable for use in medical applications on the surface of the routing guide elements 16.

The routing rollers 16 may be arranged in any of a number of configurations. The configuration of FIGS. 2 and 3 includes two routing rollers 16 being mounted distally in the housing 12 and two routing rollers 16 being mounted proximally in the housing 12. The distally mounted rollers 16 are arranged in a vertically parallel orientation with one another, and the proximally aligned rollers 16 are arranged in a horizontally parallel orientation with one another and with the opening guide element 22.

Returning to FIG. 3, the opening guide element 22 is mounted at the opening 14 of the housing 12. The opening guide element 22 provides detached mesh 20 for use in application to a wound site. The opening guide element 22 is rotatably mounted in an orthogonal orientation adjacent the peripheries of the side walls 26 such that it transverses an area near the opening 14 of the housing 12 in order to easily apply mesh 20 to a wound site. In the present embodiment, the opening guide element 22 is a roller. Further, the opening roller 22 and the plurality of routing rollers 16 may be identical and therefore interchangeable. This functionality aids in ease and convenience of manufacture and provides manufacturing cost savings.

Corresponding bosses 36 extend from the side walls 26 to provide an axle about which the opening roller 22 rotates. Similarly to the routing rollers 16, it is contemplated that an alternative conventional structure may be used as an axle for the opening roller 22.

Alternatively, the opening guide element 22 may be fixedly mounted to the housing 12, at the opening 14, provided that the surface of the opening guide element 22 is of low friction. Low surface friction may include the application of lubricants suitable for use in medical applications on the surface of the opening guide element 22.

The opening guide element 22 may be constructed of plastic, metal, or combinations thereof. For example, the opening guide element may be Teflon®, polyethylene, ultra-high molecular weight polyethylene or stainless steel.

The backing film 18 may be any suitable backing or release material used to cover the adhesive substances applied to the bottom side of the mesh 20. Such backing materials are well known in the art for covering pressure sensitive adhesives and can include, for example, paper, plastic, silicone, or the like or a composite of such materials. By way of example, the backing film 18 may be a silicone treated material. Preferably, the backing film 18 is of a material that prevents or eliminates the mesh 20 from sticking to itself when it is routed around the routing rollers 16 if any portions of the mesh 20 overlap.

The closed loop of backing film 18 is routed around at least a portion of the plurality of routing rollers 16 to define a path along which the closed loop of backing film 18 may move. In the present embodiment, the routing rollers 16 essentially act as a tractor drive system to drive or be driven by the closed loop of backing film 18. The closed loop of backing film 18 has a defined circumference, which is variable, i.e., one dispensing device 10 may include a closed loop of backing film 18 that has a circumference of 30 cm and another dispensing device 10, with identically arranged routing rollers 16, may include a closed loop of backing film 18 having a circumference of 60 cm. The routing path of the backing film 18 around the rollers 16 may change depending on the circumference of the closed loop, i.e., a loop having a relatively larger circumference may be routed in a more circuitous or convoluted path, and a loop having a relatively smaller circumference may be routed in a more direct path.

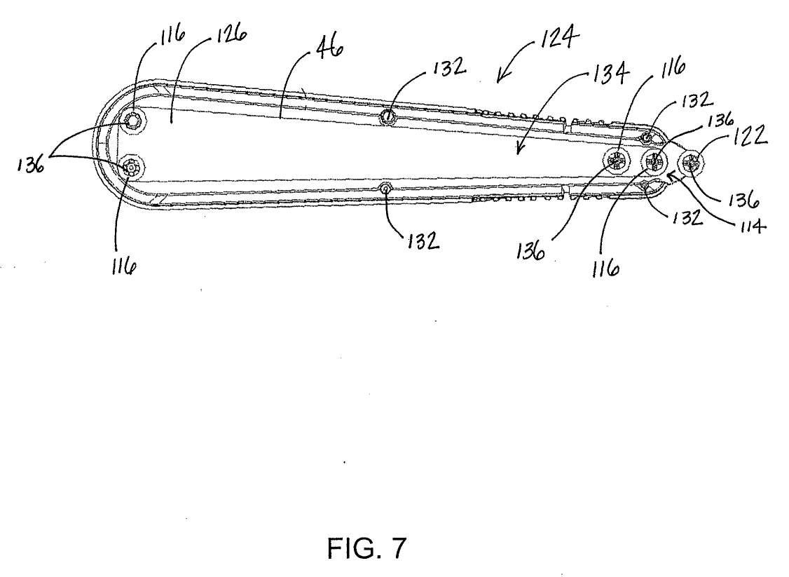

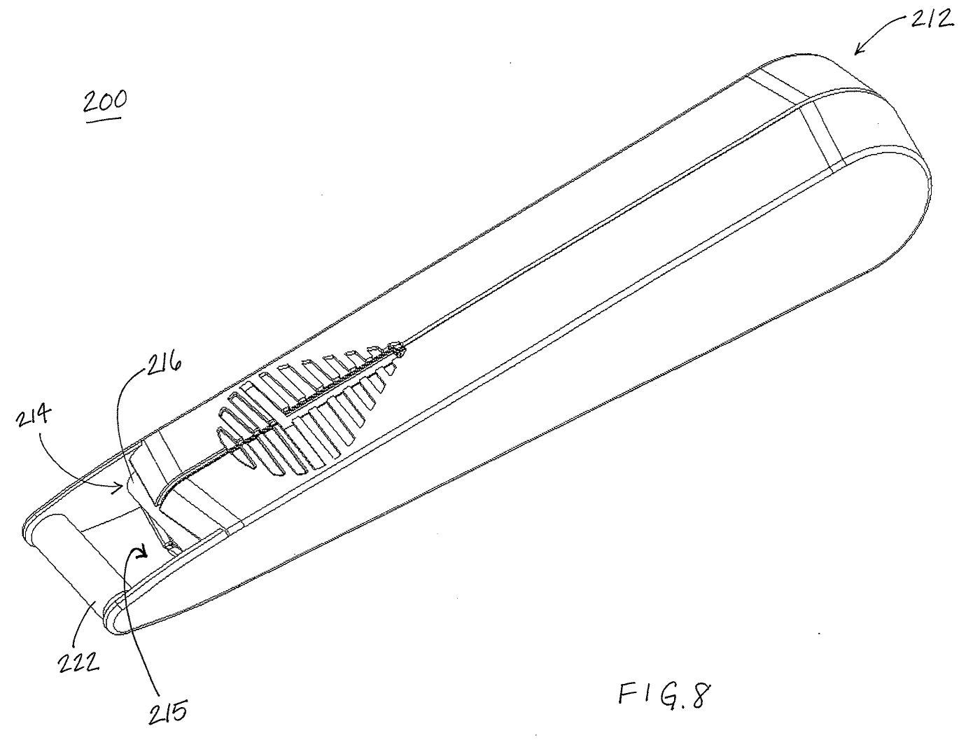

FIG. 4 shows an exemplary routing arrangement. In FIG. 4, the closed loop of backing film 18 is routed to form a convoluted V-shaped path 44 that incorporates all four of the routing rollers 16. This routing arrangement 44 would typically be used to accommodate backing films 18 having a relatively larger circumference. FIG. 7 shows an alternative exemplary routing arrangement. In FIG. 7, the closed loop of backing film is routed to form a generally triangular shape path 46 having rounded angles. This routing arrangement 46 does not incorporate all of the routing rollers 16, and would typically be used to accommodate backing films 18 having a relatively smaller circumference. In the disclosed routing arrangements 44, 46, the backing film 18 is disposed between the opening roller 22 and a forward-most routing roller 16 near the opening 14 of the housing 12. It will be understood by the ordinary artisan that the closed loop of backing film 18 may be configured in routing arrangements having various convoluted shapes, which shapes may be determined by the number of routing guide elements 16 present in the housing 12 and the number of routing guide elements 16 about which the backing film 18 is routed. For example, the backing film 18 may be routed into a W-shaped convoluted path.

Returning to FIGS. 2-4, the mesh 20 is detachably adhered to the backing film 18 via an adhesive substance applied over at least a portion of a side of the mesh 20, which side is adhered to the binding film 18. The strength of the adhesive substance on the mesh 20 is such as to hold the mesh 20 in position on a patient until a polymerizable adhesive composition may be applied (PCA). The mesh 20 typically has a defined length, which is approximately about the same as the circumference of the closed loop of binding film 18 such that portions of mesh 20 are not overlapping along the closed loop of backing film 18.

Prior to use of the dispensing device 10, substantially the entire length of mesh 20 is adhered to substantially the entire circumference of the closed loop of backing film 18 such that a combined mesh/backing film loop 48 is formed wherein the backing film portion of the combined loop 48 is closed and the mesh portion of the combined loop 48 has open ends. The combined mesh/backing film loop 48 follows the path of the underlying backing film loop 48. Accordingly, a portion of the combined mesh/backing film loop 48 is disposed between the opening roller 22 and a forward-most routing roller 16.

It is contemplated that the mesh 20 and backing film 18 will be protected within the interior 34 of the housing 12 until application of the mesh 20 begins. Means may also be provided to facilitate initial separation of the mesh 20 from the backing film 18 when applying the mesh 20 to a patient. For example, a hand leader 38 (e.g., a strip of plastic, paper, or other suitable material) may be attached to an end of the mesh 20. More specifically, an end of the hand leader 38 is attached to an end of the mesh 20 and an opposite end of the hand leader 38 is disposed exterior to the opening 14 of the housing 12 of the dispensing device 10. Preferably, the end of the hand leader 38 disposed exterior to the opening 14 is inserted into a holding space of the housing 12 or detachably connected to the housing 12 such that the hand leader 38 is not inadvertently pulled prior to use.

When the hand leader 38 is pulled, the end of mesh 20 to which it is connected is detached from its corresponding backing film 18 thereby separating a portion of the combined mesh/backing film loop 48 into constituent mesh 20 and backing film parts 18. The mesh 20 is led over the opening roller 22 by the hand leader 38, and the backing film 8 follows the path of the continuous loop of backing film 18. The force exerted on the end of the mesh 20 is transferred to the combined mesh/backing film loop 48 by the routing rollers 16 thereby advancing the loop 48 and continuing to supply detached mesh 20 to the opening roller 22 until all of the mesh 20 has been removed from the combined mesh/backing film loop 48. In this way, the opening roller 22 and the forward-most routing roller 16 facilitate detachment of the mesh 20 from the continuous loop of backing film 18. As detached mesh 20 is advanced through the opening 14, the continuous loop of backing film 18 continues to advance more detached mesh 20 to the opening 14 until all of the mesh 20 has been removed from the combined mesh/backing liner loop 48 leaving only a continuous loop of backing liner 18.

It is preferred that the mesh 20 is guided over the opening roller 22 with the adhesive side of the mesh 22 oriented away from the opening roller 22 so as to prevent or eliminate adhesion of the mesh 20 to the opening roller 22. Thus, it is preferable that the opening roller 22 is constructed or coated with a material with poor adhesion to the adhesive substance such that the adhesive substance on the mesh 20 does not adhere to the opening roller 22, compromising subsequent operation of the device 10. The opening roller 22 may present the adhesive side of the mesh 20 to a wound site and press the mesh 20 down on the wound site for promoting adhesion of the mesh 20 to the patient. While it is preferred that the dispensing device 10 include an opening roller 22, it will be clear to one of skill in the art that the opening roller 22 is not required. The dispensing device 10 operates in the same manner whether or not an opening roller 22 is present.

Preferably, the guide elements, including the opening guide element 22 and routing guide elements 16, have a low surface friction such that facile traversing of the detached mesh 20, detached backing film 18, or mesh/combined backing film loop 48 over the guide element surface is provided.

The adhesive backed mesh may be porous. By “porous” is meant herein either that the bulk of the mesh has pores, such that a subsequently applied polymerizable adhesive composition is soaked up or absorbed by the bulk material, or that the bulk of the mesh has voids (like a net or screen), such that the subsequently applied polymerizable adhesive composition passes directly through the bulk material, with or without being soaked up or absorbed by the bulk material. For example, in the case of textile materials, “porous” is generally used to mean that the applied adhesive composition permeates and passes through interstices between the fibers, but does not necessarily pass into and through the fibers themselves.

Such porosity (or other properties such as hydrophobicity or hydrophilicity) will also allow a polymerization initiator or rate modifier to be loaded in or on the mesh prior to use, to initiate the subsequently applied polymerizable adhesive composition. Such porosity will also preferably allow air and fluid to pass through the mesh, either through pores per se, or through voids in the bulk material. Depending upon the degree of porosity and/or the size of the openings, such porosity of the mesh or ability of air and fluid to permeate through the mesh may be tailored either to remain after a final composite material is formed, or to be absent therefrom. The mesh is also preferably non-toxic, as it is intended to be used to cover a wound, such as on biological tissues. As such, the mesh should be biologically compatible with the desired substrate (such as tissue, skin, organ, or the like), and is preferably a material that is governmentally approved or generally regarded as safe for the desired purpose. By way of example, suitable mesh materials are disclosed in United States Patent Applications 2006/0009099 and 2005/0182443, incorporated herein by reference in their entirety.

The mesh may be a textile or mesh/web material. Suitable textile materials may be formed of either synthetic or natural materials. Such textile material may be formed of either woven, non-woven, or knit fabrics or materials. The mesh may be, for example, any suitable polymeric film, plastic foam (including open celled foam), a woven fabric, knitted fabric, a non-woven fabric, mixture thereof, or the like. The mesh may also be a plastic film or paper film having holes formed therein. In particular, suitable mesh may be prepared, for example, from nylon, a polyolefin film, such as polyethylene, polypropylene, ethylene propylene copolymers, and ethylene butylene copolymers, polyurethanes, polyurethane foams, polystyrenes, plasticized polyvinylchlorides, polyesters, polyamides, polylactic acid, polyglycolic acid, polycaprolactone, copolymer mixtures of the above, and cotton. Suitable specific examples include, for example, nylon, polyethylene, polypropylene, ethylene propylene copolymers, ethylene butylene copolymers, polyurethane, polystyrene, plasticized polyvinylchloride, polyester, polyamide, cotton, polytetrafluoroethylene (PTFE), biovascular material, collagen, Gore-Text®, DACRON®, etc.

The mesh may be formed of a synthetic, semi-synthetic, or natural organic material. Thus, for example, the mesh may be formed of a synthetic or natural polymer material, but not from a material such as metal (such as silver, steel or the like) or glass or ceramic. The mesh is preferably resistant to tearing. The mesh may be of any thickness, however, the thickness of the mesh may typically be from about 0.1 mil to about 80 mils. In another embodiment, the thickness of the mesh is from about 0.5 mil to about 20 mils, preferably from about 0.7 mil to about 10 mils, or from about 1 mil to about 5 mils.

The mesh may be a strip from about 10 cm to about 100 cm, preferably from about 30 cm to about 60 cm in length. The mesh strip may be from about 0.1 cm to about 5 cm, preferably from about 1 cm to about 3 cm, more preferably 2 cm, in width.

The mesh may be selected to be elastic or have some memory effect. In such embodiments, the elastic properties of the mesh may desirably provide a degree of pressure or stress at the application site, for example, to maintain wound edge approximation. Likewise, in embodiments where such additional degree of pressure or stress at the application site is not desired, the mesh may be selected to have less or no elasticity.

The mesh may be either biodegradable, or non biodegradable. By “biodegradable” is meant that the mesh biodegrades over time in vivo, such that it does not require physical removal of the mesh after a set period of time. Thus, for example, a biodegradable mesh is one that, in the in vivo environment, will biodegrade over a period of from about one week to about five years. A non biodegradable material is one that does not biodegrade in an in vivo environment within about five years. Such a non biodegradable material thus would require physical removal of the mesh at a desired time, rather than slowly deteriorating over time.

The mesh may contain materials such as a polymerization initiator, accelerator, rate-modifier, and/or cross-linking agent for initiating polymerization and/or cross-linking of a polymerizable monomer material. The mesh preferably includes one or more chemical materials located in or on the mesh. For example, one or more chemical substances may be dispersed in or on the mesh, such as being chemically bound, physically bound, absorbed, or adsorbed to the mesh. Thus, for example, the mesh preferably includes at least a polymerization initiator or rate modifier, and may optionally include one or more bioactive materials. As desired, the one or more chemical substances may be either immobilized in or on the mesh, for example, so that it has a desired effect but is not detached from the mesh during use.

For example, a polymerization initiator or rate modifier may be loaded in or on the mesh so that the initiator or rate modifier provides the desired initiation or rate modification effect to a subsequently applied polymerizable adhesive composition. The polymerization initiator or rate modifier may be immobilized in or on the mesh, so that the initiator or rate modifier does not become detached from the mesh and its residues dispersed in the resultant polymeric material. Alternatively, for example, the polymerization initiator or rate modifier may be initially attached to the mesh, but only in such a manner that it becomes mobilized or solubilized by a subsequently applied polymerizable adhesive composition and dispersed in the resultant polymeric material.

If desired, a combination of chemical substances may also be provided in or on the mesh, to provide multiple effects. For example, as described above, a first chemical species (such as a polymerization initiator or rate modifier) may be immobilized in or on the mesh, while a second, different chemical species (such as a bioactive material) may be detachably attached to the mesh. Other combinations of chemical species and resultant effects are also envisioned.

When present in or on the mesh, the chemical substances (i.e., polymerization initiator, rate modifier, and/or bioactive materials, or other additives), may be incorporated in or on the mesh in any suitable manner. For example, the chemical substance may be added to the mesh by contacting the mesh with a solution, mixture, or the like including the chemical substances. The chemical substance may be added to the mesh, for example, by dipping, spraying, roll coating, gravure coating, brushing, vapor deposition, or the like. Alternatively, the chemical substance may be incorporated into or onto the mesh during manufacture of the mesh, such as during molding, knitting/weaving, scouring, tenting, plaiting or other processing or the like of the mesh.

The chemical substance may be present in or on the mesh in any suitable concentration and manner. For example, the chemical substance may be applied in a uniform manner to the mesh, such that there is a substantially uniform concentration of the chemical substance across the mesh. Alternatively, the chemical substance may be applied such that a concentration gradient exists across or through the mesh. For example, a greater or smaller concentration of the chemical substance could exist at the center or edges of the mesh, or a greater or smaller concentration of the chemical substance could be applied on one side of the mesh as compared to an opposite side. Further, the chemical substance may be applied in a uniform manner to the mesh, or it may be applied in a non-uniform random or patterned manner (such as lines, dots, concentric circles, or the like). The chemical substances may also be on, beneath, or in the pressure sensitive adhesive layer applied to the mesh.

Other chemical substances that may be present in or on the mesh include, but are not limited to, any suitable and preferably compatible additive that enhances performance of the composite structure. Such additional chemical substances may be bioactive or non-bioactive. Suitable other chemical substances thus include, but are not limited to, colorants (such as inks, dyes and pigments), scents, protective coatings that do not chemically detach, temperature sensitive agents, drugs, wound-healing agents, anti-microbial agents and the like.

The polymerization initiator or rate modifier loaded in or on the mesh may provide a number of advantages for example, the tailoring of the setting or polymerization time of the applied polymerizable adhesive composition. For example, the type and/or concentration of initiator that is applied to the mesh may be selected so as to provide faster or slower polymerization time. The concentration of polymerization initiator or rate modifier may be increased to provide a faster polymerization time, or may be decreased to provide a slower polymerization time.

Because the polymerization initiator or rate modifier is loaded directly in or on the mesh, it is not necessary to mix the polymerizable adhesive composition with a polymerization initiator or rate modifier prior to application. This may allow a longer working time, where the polymerizable monomer composition may be more precisely and carefully applied over a longer period of time. Notwithstanding the foregoing, it is possible to have a rate modifier in the adhesive applicator rather than on the mesh. Accordingly, the inclusion of a rate modifier in the adhesive applicator is within the scope of the present invention.

Such suitable initiators are known in the art and are described, for example, in U.S. Pat. Nos. 5,928,611 and 6,620,846, both incorporated herein by reference in their entireties, and U.S. Patent Application No. 2002/0037310, also incorporated herein by reference in its entirety. Quaternary ammonium chloride and bromide salts useful as polymerization initiators are particularly suitable. By way of example, quaternary ammonium salts such as domiphen bromide, butyrylcholine chloride, benzalkonium bromide, acetyl choline chloride, among others, may be used.

Benzalkonium or benzyltrialkyl ammonium halides such as benzyltrialkyl ammonium chloride may be used. When used, the benzalkonium halide may be benzalkonium halide in its unpurified state, which comprises a mixture of varying chain-length compounds, or it can be any suitable purified compound including those having a chain length of from about 12 to about 18 carbon atoms, including but not limited to C12, C13, C14, C15, C16, C17, and C18 compounds. By way of example, the initiator may be a quaternary ammonium chloride salt such as benzyltrialkyl ammonium chloride (BTAC).

Other initiators or accelerators may also be selected by one of ordinary skill in the art without undue experimentation. Such suitable initiators or accelerators may include, but are not limited to, detergent compositions; surfactants: e.g., nonionic surfactants such as polysorbate 20 (e.g., Tween 20™ from ICI Americas), polysorbate 80 (e.g., Tween 80™ from ICI Americas) and poloxamers, cationic surfactants such as tetrabutylammonium bromide, anionic surfactants such as sodium tetradecyl sulfate, and amphoteric or zwitterionic surfactants such as dodecyldimethyl(3-sulfopropyl)ammonium hydroxide, inner salt; amines, imines and amides, such as imidazole, arginine and povidine; phosphines, phosphites and phosphonium salts, such as triphenylphosphine and triethyl phosphite; alcohols such as ethylene glycol, methyl gallate; tannins; inorganic bases and salts, such as sodium bisulfite, calcium sulfate and sodium silicate; sulfur compounds such as thiourea and polysulfides; polymeric cyclic ethers such as monensin, nonactin, crown ethers, calixarenes and polymeric-epoxides; cyclic and acyclic carbonates, such as diethyl carbonate; phase transfer catalysts such as Aliquat 336; organometallics such as cobalt naphthenate and manganese acetylacetonate; and radical initiators or accelerators and radicals, such as di-t-butyl peroxide and azobisisobutyronitrile.

Mixtures of two or more, such as three, four, or more, initiators or accelerators may be used. A combination of multiple initiators or accelerators may be beneficial, for example, to tailor the initiator of the polymerizable monomer species. For example, where a blend of monomers is used, a blend of initiators may provide superior results to a single initiator. For example, the blend of initiators can provide one initiator that preferentially initiates one monomer, and a second initiator that preferentially initiates the other monomer, or can provide initiation rates to help ensure that both monomer species are initiated at equivalent, or desired non-equivalent, rates. In this manner, a blend of initiators can help minimize the amount of initiator necessary. Furthermore, a blend of initiators may enhance the polymerization reaction kinetics. The polymerization initiator, accelerator, rate-modifier, and/or cross-linking agent may be incorporated into the mesh using impregnation methods known in the art.

The adhesive substance used in the mesh may, for example, be any suitable adhesive substance. Preferably, the adhesive substance is a medical grade adhesive, such as acrylic based pressure sensitive adhesives (PSAs), rubber based pressure sensitive adhesives, silicone pressure sensitive adhesives, mixtures thereof, or the like. It is preferred that the adhesive substance be different from the polymerizable adhesive composition. Thus, for example, it is preferred that while the polymerizable adhesive composition can be, for example, a polymerizable monomeric adhesive composition, the adhesive substance is a material that is not a polymerizable adhesive composition, such as a pressure sensitive adhesive.

Suitable rubber based PSAs include, but are not limited to, those taught in U.S. Pat. No. 5,705,551 and in U.S. Pat. No. 4,080,348, the disclosures of which are hereby incorporated by reference. Examples of polymeric rubber bases include one or more of styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene, and styrene-butadiene rubber.

A particularly useful rubber based adhesive is that which has a thermoplastic elastomeric component and a resin component. The thermoplastic elastomeric component contains about 55-85 parts of a simple A-B block copolymer wherein the A-blocks are derived from styrene homologs and the B-blocks are derived from isoprene, and about 15-45 parts of a linear or radical A-B-A block copolymer wherein the A-blocks are derived from styrene or styrene homologs and the B-blocks are derived from conjugated dienes or lower alkenes, the A-blocks in the A-B block copolymer constituting about 10-18 percent by weight of the A-B copolymer and the total A-B and A-B-A copolymers containing about 20 percent or less styrene. The resin component consists of essentially of tackifier resins for the elastomeric component. In general any compatible conventional tackifier resin or mixture of such resins may be used. These include hydrocarbon resins, rosin and rosin derivatives, polyterpenes and other tackifiers. The adhesive substance may contain about 20-300 parts of the resin component per one hundred parts by weight of the thermoplastic elastomeric component. One such rubber based adhesive substance is commercially available from Ato Findley under the trade name HM3210.

Useful acrylic based PSAs include, but are not limited to, those taught in U.S. Pat. No. 5,947,917 and U.S. Pat. No. 5,164,444 (acrylic emulsion), U.S. Pat. No. 5,623,011 (tackified acrylic emulsion). It can also be radiation curable mixture of monomers with initiators and other ingredients such as those taught in U.S. Pat. No. 5,232,958 (UV cured acrylic) and U.S. Pat. No. 5,232,958 (EB cured). The disclosures of these patents are hereby incorporated by reference.

It is contemplated that any acrylic based polymer capable of forming an adhesive layer with sufficient tack to adhere to the mesh, the backing film or to a substrate, and with acceptable adhesion to skin, may be used. In certain embodiments, the acrylic polymers for the pressure-sensitive adhesive layers include those formed from polymerization of at least one alkyl acrylate monomer or methacrylate, an unsaturated carboxylic acid and optionally a vinyl lactam. Examples of suitable alkyl acrylate or methacrylate esters include, but are not limited to, butyl acrylate, ethyl acrylate, 2-ethylhexyl acrylate, isooctyl acrylate, isononyl acrylate, isodecyl acrylate, methyl acrylate, methylbutyl acrylate, 4-methyl-2-pentyl acrylate, sec-butyl acrylate, ethyl methacrylate, isodecyl methacrylate, methyl methacrylate, and the like, and mixtures thereof. Examples of suitable ethylenically unsaturated carboxylic acids include, but are not limited to, acrylic acid, methacrylic acid, fumaric acid, itaconic acid, and the like, and mixtures thereof. A preferred ethylenically unsaturated carboxylic acid monomer is acrylic acid. Examples of suitable vinyl lactams include, but are not limited to, N-vinyl caprolactam, 1-vinyl-2-piperidone, 1-vinyl-5-methyl-2-pyrrolidone-, vinyl pyrrolidone, and the like, and mixtures thereof.

Useful silicone pressure sensitive adhesives include those commercially available from Dow Corning Corp., Medical Products and those available from General Electric. Examples of silicone adhesives available from Dow Corning include those sold under the trademarks BIO-PSA X7-3027, BIO-PSA X7-4919, BIO-PSA X7-2685, BIO-PSA X7-3122 and BIO-PSA X7-4502. Additional examples of silicone pressure sensitive adhesives are described in U.S. Pat. Nos. 4,591,622, 4,584,355, 4,585,836 and 4,655,767, the entire disclosures of which are incorporated herein by reference.

The adhesive substance may also include one or more tackifiers, plasticizers, antioxidants, cutting agents such as waxes, and surfactants. Other optional materials that may be added to the adhesive substance layer in minor amounts (typically less than about 25% by weight of the elastomeric phase) include pH controllers, medicaments, bactericides, growth factors, wound healing components such as collagen, antioxidants, deodorants, perfumes, antimicrobials and fungicides.

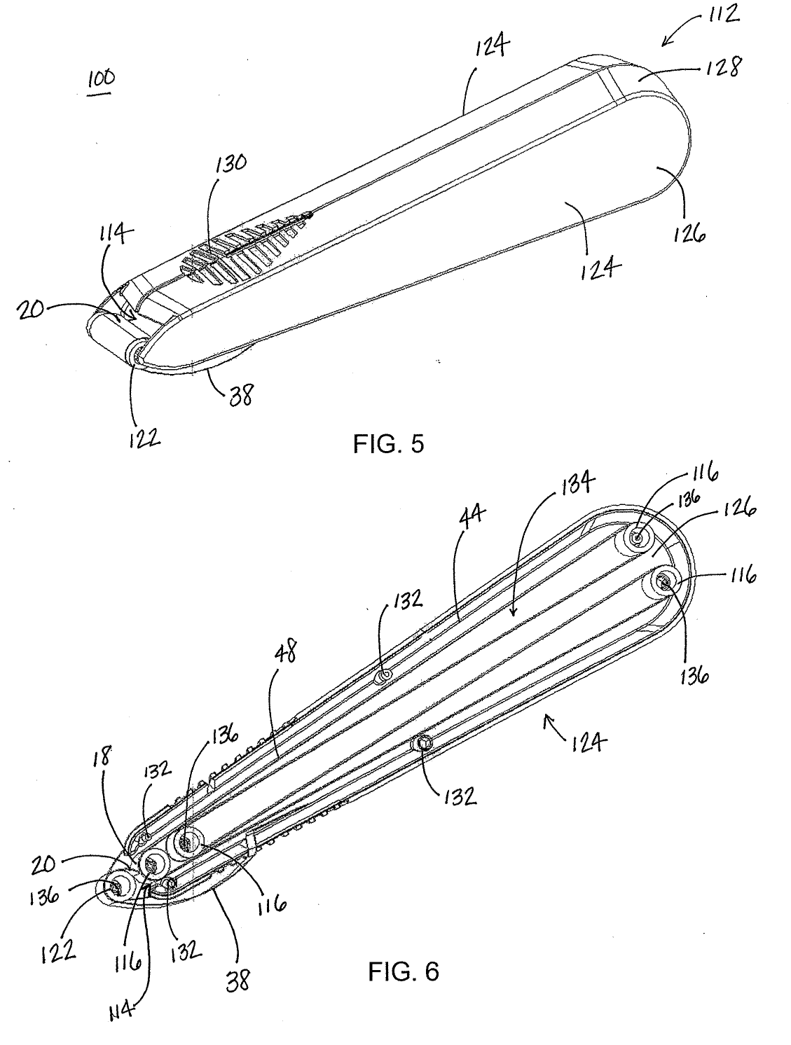

Returning to the figures, FIGS. 5-7 show an alternative embodiment of the dispensing device. More particularly, FIG. 5 is a perspective view of the dispensing device; FIG. 6 is a perspective view of the dispensing device with a side member removed in order to show the internal components of the dispensing device, and FIG. 7 is a top plan view of the dispensing device with the combined mesh/backing film loop being routed in a configuration that does not include all of the routing rollers.

Similarly to dispensing device 10, the mesh dispensing device 100 comprises an ergonomically shaped housing 112 having an opening 114 formed in a proximal end thereof, a plurality of routing guide elements 116 mounted in the housing 112, a closed loop of backing film 18 routed around at least a portion of the plurality of routing guide elements 116, mesh 20 detachably adhered to the closed loop of backing film 18; and optionally, an opening guide element 112 mounted at the opening 114 of the housing 112.

Most of the components of the dispensing device 100 are the same as or very similar to the dispensing device 10. With further regard to the dispensing device 100, the housing 112 comprises two side members 124 that are joined to form the housing 112. Each side member 124 includes a side wall 126 and a perimeter wall 128 extending around and away from a periphery of the side wall 126. The perimeter wall 128 extends substantially completely around the periphery of the side wall 128 except for a spaced area in the perimeter wall 128 that forms the opening 114 of the housing 112 with the spaced area of the other side member 124 when the side members 124 are joined. Ridges 130 may be formed in portions of the perimeter wall 128 and/or portions of the side wall 126 for improving a clinician's ability to hold the device 100 during use. In addition, other contoured indentations (not shown) may be included in the side walls 126 or perimeter walls 128 of the housing 112.

The housing 112 of dispensing device 100 is relatively smaller than the housing 12 of dispensing device 10. It is noteworthy that differently sized and shaped housings 12, 112 may contain the same internal components, including the same length of mesh 20. Advantageously, this enables a clinician to choose a dispensing device 10, 100 based on ease and comfort of use without sacrificing functionality and mesh length. One clinician may prefer a smaller more compact housing 112, while another may prefer a more robust housing 12.

With reference to FIGS. 6 and 7, as with the dispensing device 10, mating connection members 132 project orthogonally from the side walls 126 of the housing 112 into an interior 134 of the housing 112. In the present embodiment, the mating connection members 132 are projections that fit together in a friction fit to join the side members 124 thereby forming the housing 112. As such, the projection 132 of one side member 124 consists of a solid projection member and the mating projection 132 of the other side member consists of a sleeve into which the solid projection is inserted in a friction fit.

The plurality of routing guide elements 116 are mounted to the side members 124 of the housing 112 in orthogonal relation thereto such that the routing guide elements 116 transverse the interior 134 of the housing 112. In the present embodiment, the routing guide elements 116 are rotatably mounted rollers. Corresponding bosses 136 extending from the side walls 126 of the housing 112 serve as axles about which the routing rollers rotate 116. Such arrangement ensures that each of the routing rollers 116 is operatively connected to each of the side members 124. The present embodiment includes four rollers. The routing rollers 116 may be arranged in any of a number of configurations. The configuration of FIGS. 6 and 7 includes two routing rollers 116 being mounted distally in the housing 112 and two routing rollers 116 being mounted proximally in the housing 112. The distally mounted routing rollers 116 are arranged in a vertically parallel orientation with one another, and the proximally aligned routing rollers 116 are arranged in a horizontally parallel orientation with one another and with the opening guide element 122.

The opening guide element 122 is mounted near the opening 114 of the housing 112. The opening guide element 122 provides detached mesh 20 for use in application to a wound site. The opening guide element 122 is rotatably mounted in an orthogonal orientation adjacent the peripheries of the side walls 126 such that it transverses an area near the opening 114 of the housing 112 in order to easily apply mesh 20 to a wound site. In the present embodiment, the opening guide element 122 is a roller. Further, the opening roller 122 and the plurality of routing rollers 116 may be identical and therefore interchangeable. This functionality aids in ease and convenience of manufacture and provides manufacturing cost savings.

Corresponding bosses 136 extend from the side walls 126 to provide an axle about which the opening roller 122 rotates. Similarly to the routing rollers 116, it is contemplated that an alternative conventional structure may be used as an axle for the opening roller 122.

The backing film 18 may be any suitable backing or release material used to cover the adhesive substances applied to the bottom side of the mesh 20. The closed loop of backing film 18 is routed around at least a portion of the plurality of routing rollers 116 to define a path along which the closed loop of backing film 18 may move. The routing rollers 116 essentially act as pulleys that drive or are driven by the closed loop of backing film 18. The closed loop of backing film 18 has a defined circumference, which is variable, i.e., one dispensing device 100 may include a closed loop of backing film 18 that has a circumference of 30 cm and another dispensing device 100, with identically arranged routing rollers 116, may include a closed loop of backing film 18 having a circumference of 60 cm. The routing path of the backing film 18 around the rollers 116 may change depending on the circumference of the closed loop, i.e., a loop having a relatively larger circumference may be routed in a more circuitous or convoluted path, and a loop having a relatively smaller circumference may be routed in a more direct path.

FIG. 6 shows an exemplary routing arrangement. In FIG. 6, the closed loop of backing liner is routed to form a convoluted V-shaped path 44 that incorporates all four of the routing rollers 116. This routing arrangement 44 would typically be used to accommodate backing liners 18 having a relatively larger circumference. FIG. 7 shows an alternative exemplary routing arrangement. In FIG. 7, the closed loop of backing film is routed to form a generally triangular shaped path 46 having rounded angles. This routing arrangement 46 does not incorporate all of the routing rollers 116, and would typically be used to accommodate backing liners 18 having a relatively smaller circumference. In the disclosed routing arrangements 44, 46, the backing film 18 is disposed between the opening roller 122 and a forward-most routing roller 116 near the opening of the housing.

The mesh 20 used in device 100 may be the same as that used in device 10.

Prior to use of the dispensing device 100, substantially the entire length of mesh 20 is adhered to substantially the entire circumference of the closed loop of backing film 18 such that a combined mesh/backing film loop 48 is formed wherein the backing film 18 portion of the combined loop 48 is closed and the mesh portion of the combined loop 48 has open ends. The combined mesh/backing film loop 48 follows the path of the underlying backing film loop 48. Accordingly, a portion of the combined mesh/backing film loop 48 is disposed between the opening roller 122 and a forward-most routing roller 116.

It is contemplated that the mesh 20 and backing film 18 will be protected within the interior 134 of the housing 112 until application of the mesh 20 begins. A hand leader 38 may be attached to an end of the mesh 20.

FIG. 8 is a perspective view of a second alternative embodiment of the dispensing device 200. Similarly to dispensing devices 10, 100, the mesh dispensing device 200 comprises an ergonomically shaped housing 212 having an opening 214 formed in a proximal end thereof, a plurality of routing guide elements 216 mounted in the housing 212, a closed loop of backing film (not shown) routed around at least a portion of the plurality of routing guide elements 216, mesh (now shown) detachably adhered to the closed loop of backing film; and optionally, an opening guide element 222 mounted near the opening 214 of the housing 212.

Many of the components of the dispensing device 200 are the same as or similar to the components of the previously described dispensing devices 10,100. However, a noteworthy difference between the dispensing device 200 and the dispensing devices 10, 100 is the spacing between the opening guide roller 222 and a forward-most routing roller 216. The amount of space between the opening roller 222 and forward-most routing roller 216 creates a viewing window 215 through which a clinician may see the mesh being applied to a wound site.

In previous mesh dispensing devices, it was necessary to minimize the length of unsupported mesh, i.e., mesh length after separation from the backing film until application to a wound site, to avoid deformation of the mesh prior to application to the wound site. These dispensing devices use a supply spool for providing the combined mesh and backing film and a take-up spool for storing detached backing film. In addition, a clutching mechanism is used to minimize slack in the backing film caused by using a supply and take-up spool. The axial loads created by the supply spool, take-up spool, and clutching mechanism in combination with the elastomeric nature of the mesh caused the mesh to be deformed if the length of unsupported mesh was too long. An exemplary dispensing device of this type is described in commonly assigned U.S. patent application Ser. No. 11/759,774, which is hereby incorporated by reference in its entirety. The instant dispensing device uses a continuous loop of backing film rather than a supply spool, take-up spool, and clutching mechanism, therefore, the axial loads caused thereby are not present in the dispensing device.

The viewing window 215 is advantageous because it enables a clinician using the device 200 with a pushing motion to see whether the mesh is being applied in a manner in which he or she desires almost instantaneously with its application. Without the window 215, in a pushing application, the clinician must wait until the device 200 has cleared the wound site before he or she can see whether the mesh has been applied in a manner in which he or she desires. Accordingly, the viewing window 215 of the dispensing device 200 provides the advantage of being able to see the applied mesh more quickly.

To use the dispensing device 10, 100, 200, the outer end of the hand leader 38 is freed from the housing 12, 112, 212 and pulled towards the rear of mesh dispensing device 10, 100, 200. The hand leader 38 initiates detachment of the mesh 20 from the backing film 18 and routes the detached mesh 20 to the opening 14, 114, 214 with the adhesive side of the mesh 20 presented for use. The mesh dispensing device 10, 100, 200 is positioned at a wound site, for example, near the end or edge of the wound. The mesh 20 initially presented from the dispensing device 10, 100, 200 is adhered to the wound site via PSA on the mesh 20 starting at a region of the wound line proximal to the dispensing device 10, 100, 200, and the mesh dispensing device 10, 100, 200 is then pushed along the wound line towards a distal region of the wound line so the mesh 20 initially passes under and later behind the dispensing device 10, 100, 200 as the dispensing device 10, 100, 200 is moved from the proximal region of the wound line to the distal region of the wound line. If mesh 20 is applied using a pulling motion, the mesh 20 initially presented from the dispensing device 10, 100, 200 is adhered to a distal region of the wound line, and the device 10, 100, 200 is then pulled along the wound line toward a proximal region of the wound line. Mesh 20 and backing film 18 are routed between the opening roller 22, 122, 222 and a forward-most routing roller 16, 116, 216 that may provide a separation point for the mesh 20 and backing film 18 in cooperation with the mesh 20 adhering to the wound site, directing the mesh 20 out of the device 10, 100, 200 to the wound site while the backing film 18 is maintained in the closed loop. A clinician continues to push or pull the dispensing device 10, 100, 200 until the mesh 20 loaded in the device 10, 100, 200 has been used. In this manner, tissue edge approximation may be performed concurrent with controlled placement of the mesh 20. The detached backing film 18 remains in the device 10, 100, 200 in a closed loop. A clinician may simply dispose the depleted device 10, 100, 200 after use and start with a new device 10, 100, 200 if more mesh 20 is needed.

After applying the mesh to a wound site, a clinician may also apply a polymerizable adhesive composition over, i.e. substantially covering, at least a portion of the mesh and allow the polymerizable adhesive composition to permeate into and under the mesh and polymerize to form a composite structure bonded to the tissue surface. The polymerizable adhesive composition may comprise a polymerizable monomeric adhesive. In embodiments, the polymerizable adhesive composition comprises a polymerizable 1,1-disubstituted ethylene monomer formulation. In embodiments, the polymerizable adhesive composition comprises a cyanoacrylate formulation. In embodiments, synthetic polymerizable adhesive materials such as polyurethane, polyethylene glycol, acrylates, glutaraldehyde and biologically based adhesives may be used.

Suitable α-cyanoacrylate monomers which may be used, alone or in combination, include alkyl α-cyanoacrylates such as 2-octyl cyanoacrylate; dodecyl cyanoacrylate; 2-ethylhexyl cyanoacrylate; butyl cyanoacrylate such as n-butyl cyanoacrylate; ethyl cyanoacrylate; methyl cyanoacrylate or other α-cyanoacrylate monomers such as methoxyethyl cyanoacrylate; 2-ethoxyethyl cyanoacrylate; 3-methoxybutyl cyanoacrylate; 2-butoxyethyl cyanoacrylate; 2-isopropoxyethyl cyanoacrylate; and 1-methoxy-2-propyl cyanoacrylate. In embodiments, the monomers are ethyl, n-butyl, or 2-octyl a-cyanoacrylate. Other cyanoacrylate monomers which may be used include alkyl ester cyanoacrylates, such as those prepared by the Knoevenagel reaction of an alkyl cyanoacetate, or an alkyl ester cyanoacetate, with paraformaldehyde, subsequent thermal cracking of the resultant oligomer and distillation.

Composite products comprised of a tissue bonding material, such as an adhesive backed mesh, for example, used in conjunction with a polymerizable adhesive composition may be used for tissue bonding. Use of the aforementioned material requires that the mesh be adhered to a substrate, for example a patient's skin or tissue, straddling well-approximated wound edges. The polymerizable adhesive composition, for example, a cyanoacrylate (CA) adhesive may then be applied over the mesh after it is applied to a patient, further securing the mesh in place and promoting wound closure. The adhesive backed mesh holds the wound edges in place prior to application of the CA adhesive, and may provide a matrix for supporting initiating chemicals that modulate the curing rate of the CA adhesive and may also provide mechanical reinforcement of the CA adhesive.

To work effectively, the adhesive backed mesh preferably should be in intimate contact with the patient's skin over its entire surface. “Tenting” of the mesh (e.g., air gaps between the patient's skin and the mesh) may result in gaps that do not fill-in when the CA adhesive is applied, resulting in a discontinuous closure and/or area of weak adhesion, which may lead to premature separation of the incision. Furthermore, the surface to which the mesh is being applied may be undulating in its topography (e.g., being curved in 3-dimensions). To address these requirements, the adhesive backed mesh is typically flexible and compliant.

The adhesive backed mesh preferably should be precisely applied to the patient's skin such that the wound line is centered along the axis of the mesh strip as to evenly distribute the load on either side of the wound after completing application of the mesh strip (for approximating opposite sides of the wound line, for example). Lastly, the adhesive backed mesh preferably should hold the wound edges in alignment prior to application of the CA adhesive to ensure a desirable cosmetic outcome.

The dispensing device provides for a one-handed means of applying a long segment of PSA backed surgical mesh to a patient over an undulating path with minimal distortion to the mesh while providing clear visualization of the target trajectory. This also minimizes the complexity of the mechanisms, provides for easier assembly, and is designed for manufacturability and cost savings (i.e. one component is used for both housing halves.) The presented embodiments provide for means of carrying various length of mesh for different application needs.

The dispensing device herein disclosed provides for the surface of the adhesive backed mesh, which ultimately adheres to the substrate to which it is being applied, being oriented toward a backing film. Moreover, the dispensing device herein disclosed reduces or eliminates the tensile load required to pay the tape from the dispensing device, which may otherwise result in the mesh distorting and/or elongating and may lead to undesirable tenting of the mesh, for example. Additionally, the dispensing device herein disclosed is operable in both a forward pushing mode and a pulling mode. The forward mode allows visualization of the substrate immediately ahead of where the mesh is to be applied. Such a mode of operation may be ideal for a surgical setting where the clinician may need to follow an irregularly-shaped wound that may traverse over undulating contour. Such advantages are not collectively addressed in dispensing devices designed for conventional adhesive tape and other materials not directed to use in tissue bonding.

The device herein disclosed is adapted for use with materials that can be applied to a surface, and impregnated with a polymerizable adhesive composition, which upon setting or curing provides an adherent structure over the surface. For example, any adhesive backed mesh (or adhesive backed flexible material) suitable for use in the dispensing device disclosed herein includes materials suitable for tissue bonding. Suitable flexible materials include, for example, those described in co-assigned U.S. Patent Application Publication No. 2006/0009099, incorporated herein by reference in its entirety. The mesh is preferably flexible or compliant, to allow the mesh to be placed on the desired surface (such as skin, organ, tissue, or the like) in a manner that allows the mesh to conform to the topology of the desired surface. Likewise, the mesh is preferably porous, to allow a subsequently applied polymerizable adhesive composition to pass through or permeate through the mesh and to polymerize, while adhering the mesh to the desired surface. A flexible material such as a film or tape may also be dispensed in the dispensing device described herein instead of a mesh.

A device for dispensing a mesh for purposes of tissue bonding is provided. Using the device as herein disclosed, application of the mesh is achieved without distortion of the mesh, allowing conforming to undulating/curved topography. Using the device as herein disclosed, application of the mesh may be achieved without compromise of the clinician's visualization of the wound edge during application.

The device herein disclosed allows for a one-handed mesh placement operation adaptable to long pieces of an adhesive backed mesh. The device may allow use of gloved hands and for the user to more accurately approximate a wound edge, for example, over an undulating path with minimal distortion to the mesh while providing clear visualization of the target trajectory.

The device is configured such that the mesh is deployed to the tissue while pushing or pulling the dispensing device forward. In this manner, the clinician is presented with a clear view of the wound line on the tissue and is able to provide that the mesh substantially remains centered about this wound line as it is being applied.

The mesh dispensing device herein disclosed may be provided in a kit comprising additional components. The kit may comprise at least one mesh dispensing device as herein described, and one or more containers of polymerizable adhesive composition. The different components or groups of components may be sterilized in separate containers before packaging the components or groups of components within a kit, and thereafter sterilizing the kit as disclosed in co-assigned U.S. Pregrant Patent Publication No. 2004/0120849, incorporated herein by reference in its entirety. The kit may include one or more polymerizable adhesive compositions.

The present mesh dispensing device may be modified without departing from the spirit and scope of the present mesh dispensing device. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims.

We claim:

1. A mesh dispensing device comprising:
(a) a housing having a substantially enclosed interior and an opening formed therein;
(b) a plurality of routing guide elements mounted transversely in the interior of the housing;
(c) a closed loop of backing film having a defined circumference, the closed loop of backing film being routed around at least a portion of the plurality of routing guide elements to define a path along which the closed loop of backing film moves;
(d) a defined length of mesh comprising an adhesive substance applied over at least a portion of a side of the mesh, the mesh being detachably adhered to the closed loop of backing film via the adhesive side of the mesh; and
(e) an opening guide element mounted transversely near the opening of the housing, the opening guide element receiving mesh that has been detached from the closed loop of backing film, such that the adhesive side of the mesh is presented at the opening of the housing for dispensing.

2. The mesh dispensing device of claim 1, wherein the opening guide element consists of a roller rotatably mounted at the opening, the rotatably mounted roller of the guide element cooperating with a guide element of the plurality of routing guide elements to separate the mesh from the closed loop of backing film and present the adhesive side of the mesh at the opening of the housing.

3. The mesh dispensing device of claim 1, wherein the plurality of routing guide elements are rotatably mounted rollers.

4. The mesh dispensing device of claim 1, wherein spacing between the opening guide element and the plurality of routing guide elements is sufficient to enable a viewing window to be formed in the housing between the opening guide element and the plurality of routing guide elements.

5. The mesh dispensing device of claim 1, wherein the plurality of routing guide elements and the opening guide element consist of rollers that are the same size such that they are interchangeable.

6. The mesh dispensing device of claim 1, wherein the defined circumference of the closed loop of backing film is approximately about the same as the defined length of mesh.

7. The mesh dispensing device of claim 1, wherein the plurality of routing guide elements consists of four routing guide elements.

8. The mesh dispensing device of claim 1, wherein the housing includes two interchangeable side members that are joined to form the housing.

9. A mesh dispensing device comprising:
(a) a housing having an opening formed therein;
(b) a plurality of routing guide elements mounted transversely in the housing;
(c) a closed loop of backing film having a defined circumference, the closed loop of backing film being routed around at least a portion of the plurality of routing guide elements to define a convoluted path along which the closed loop of backing film moves; and
(d) a defined length of mesh comprising an adhesive substance applied over at least a portion of a side of the mesh, the mesh being detachably adhered to the closed loop of backing film via the adhesive side of the mesh;
(e) wherein the mesh is separated from the closed loop of mesh and backing film such that the adhesive side of the separated mesh is presented to the opening of the housing for dispensing.

10. The mesh dispensing device of claim 9, wherein the convoluted path consists of a V-shaped path.

11. The mesh dispensing device of claim 9, wherein the plurality of routing guide elements consist of rotatably mounted rollers.

12. The mesh dispensing device of claim 1 1, wherein each roller of the plurality of rollers is the same size such that the rollers are interchangeable.

13. The mesh dispensing device of claim 9, wherein the defined circumference of the closed loop of backing film is approximately about the same as the defined length of mesh.

14. The mesh dispensing device of claim 9, wherein the closed loop of backing film is routed around all of the plurality of routing guide elements.

15. The mesh dispensing device of claim 9, comprising four routing guide elements.

16. The mesh dispensing device of claim 9, wherein the housing includes two interchangeable side members that are assembled to form the housing.

17. A method for tissue bonding comprising
(a) providing a dispensing device, the dispensing device comprising:
(i) a housing having an opening formed therein;
(ii) a plurality of routing guide elements mounted transversely in the housing;
(iii) a closed loop of backing film having a defined circumference, the closed loop of backing film being routed around at least a portion of the plurality of routing guide elements to define a path along which the closed loop of backing film moves;
(iv) a defined length of mesh comprising a pressure sensitive adhesive substance applied over at least a portion of a side of the mesh, the mesh being detachably adhered to the closed loop of backing film via the adhesive side of the mesh; and
(v) an opening guide element mounted transversely in the housing, the opening guide element receiving mesh that has been separated from the closed loop of backing film, such that the adhesive side of the mesh is presented at the opening of the housing for dispensing.
(b) contacting a tissue surface in need of bonding, wherein at least a portion of the pressure sensitive adhesive side of the mesh is positioned between the tissue surface and the opening guide element;
(c) adhering the at least a portion of the pressure sensitive adhesive side of the mesh to the tissue surface;
(d) operating the dispensing device along the tissue surface;
(e) dispensing the mesh from the opening of the device; and
(f) bonding the tissue surface.

18. The method of claim 17, wherein the mesh is impregnated with a polymerization initiator.

19. The method of claim 17, wherein the step of bonding the tissue surface by dispensing the mesh from the opening of the dispensing device along the tissue surface further comprises approximating the tissue surface in need of bonding.

20. The method of claim 17, further comprising the steps of applying a polymerizable adhesive composition over and substantially covering at least a portion of the mesh; and allowing the polymerizable adhesive composition to permeate into and under the mesh and polymerize to form a composite structure bonded to the tissue surface.
